(19) United States
(12) Patent Application Publication
(10) Pub. No.: US 2017/0325291 A1
(43) Pub. Date: Nov. 9, 2017

(54) BIOHAZARD NEUTRALIZING BAG, SYSTEM AND METHOD

(71) Applicant: Mercer Technologies, Limited, Auckland (NZ)

(72) Inventors: Alan Reg DOWMAN, Auckland (NZ); Terry Dean KEMP, Queensland (AU)

(21) Appl. No.: 15/522,999

(22) PCT Filed: Oct. 30, 2015
(86) PCT No.: PCT/NZ2015/050180

§ 371 Date: Apr. 28, 2017

(30) Foreign Application Priority Data
Oct. 31, 2014 (NZ) 701602

(51) Int. Cl. H05B 3/34; A61L 2/20; A61L 2/04

(52) U.S. Cl. CPC H05B 3/34; A61L 2/20; A61L 2/04; H05B 2203/013; H05B 2203/003

(57) ABSTRACT

A sealable biohazard neutralizing bag formed of a flexible impermeable material capable of withstanding a neutralization process performed within the bag. The bag may include a vapor generator which, when activated, heats a fluid within the bag body to generate vapor to neutralize the contents of the bag. Alternatively a portable vapor generator may supply vaporized sterilant to the bag via a sealable port. Neutralization may be performed by: a. placing an item to be neutralized within the sealable bag; b. sealing a sealable opening of the bag; c. evacuating the bag via an outlet; and d. actuating the vapor generator to generate vapor such as to produce conditions sufficient to neutralize the item.

### FIELD OF THE INVENTION

The present invention relates to a biohazard bag and a method for neutralizing biohazards within the bag so as to facilitate transport or otherwise reduce the risk posed by the biohazardous material. The bag may find application in developing countries with limited infrastructure, military field hospitals, laboratories or for infectious disease control within first world hospitals.

### BACKGROUND OF THE INVENTION

In the event of a localized or global epidemic or pandemic patients or suspected patients are typically quarantined and isolated. Items used in contact with the patent during care in isolation are deemed a biohazard and require careful collection and disposal to prevent the infection spreading to other people.

Biohazard waste packaging, transport and disposal methods currently have a number of issues and shortfalls. Quarantine and containment requires that all “Category A” infectious waste produced in the containment area should remain in the area until rendered inert or “neutralized”. Typical waste generated includes medical equipment, sharps, intravenous tubing, endoscopes, linen, and used healthcare products, Personal Protection Equipment such as gowns, masks, gloves and booties as well as by-products of cleaning that is either infected or suspected of being infected. Very few isolation facilities have sterilizers or incineration facilities that are directly coupled to the facility. In almost all cases the waste must be transported to a facility to be disposed of. Even if this facility is on-site it generally entails moving the contaminated waste through public areas with the risk of transference of infection. The CDC says that “Category A” waste (e.g. Ebola associated or Smallpox or Anthrax) that has been appropriately incinerated, autoclaved, or otherwise inactivated is not infectious, does not pose a health risk, and is not considered to be regulated medical waste or a hazardous material under Federal law. Therefore, such waste is no longer considered a Category A infectious substance and is not subject to the requirements of the HMR.

Hospital sterilizers do not filter or retain vapor and condensate prior to sterilizing and so would potentially release infectious contamination into the atmosphere during any pre sterilizing phases. These sterilizers are typically steam sterilizers that rely entirely on heat sterilization at temperatures of at least 115 degrees Celsius for at least 40 minutes. These sterilizers are bulky and expensive and require sophisticated infrastructure often not present at the site of outbreaks.

Disposal protocols dictate the procedure for bagging waste in a biohazard bag then tying or otherwise closing the bag prior to putting into another bag or two to end up with a double or triple bagged item along with multiple applications of a disinfectant spray.

This is then taken from the containment area and transported to a destruction facility. There is a high risk associated with this removal and transportation and indeed the act of removing the contamination subverts the idea of containment.

In cases of aggressive outbreaks of a pandemic the usual medical waste transport haulers tend to refuse to accept this Class A waste and it piles up within the containment area with no viable alternative solution.

Methods utilising only chemical sterilants may be ineffective for certain biohazards or may result in the sterilized package containing chemically hazardous materials that are again classified as hazardous and difficult to ship to point of disposal. Such techniques typically do not remove air from a bag and so may be unsuitable for neutralizing porous loads or loads with entrapped air. Further, such methods do not produce an evacuated neutralized bag and so are more bulky and are prone to rupture.

Thus present solutions are either too bulky and are not easily transported, expensive, ineffective, complex, require sophisticated infrastructure or do not filter or contain vapor and condensate prior to sterilizing.

It is an object of the present invention to provide a biohazard bag and a method for neutralizing biohazards within a bag that overcomes the problems of the prior art or which at least provides the public with a useful choice.

### SUMMARY OF THE INVENTION

There is thus provided a biohazard neutralizing bag including:
a. a bag body formed of a flexible impermeable material capable of withstanding a neutralization process performed within the bag having a sealable opening sized to enable items to be neutralized to be positioned within the bag body; and
b. a vapor generator which, when activated, heats a fluid within the bag body to generate vapor to neutralize the contents of the bag.

There is further provided a method of neutralizing one or more items comprising:
a. placing one or more items to be neutralized within a sealable bag having a sealable opening for receiving items to be neutralized, an outlet for the removal of fluid and an integrated sterilant vapor generator;
b. sealing the sealable opening;
c. evacuating the bag via the outlet; and
d. actuating the vapor generator to generate vapor such as to produce conditions sufficient to neutralize the one or more items.

There is also provided a biohazard neutralizing system
a. a bag body formed of a flexible material capable of withstanding a steam sterilization process performed within the bag having:
   i. a sealable opening sized to enable items to be neutralized to be positioned within the bag body; and
   ii. at least one port for evacuation of the bag and the supply of vaporized sterilant; and
b. a portable vapor generator adapted to supply vaporized sterilant to the bag via the sealable port.

Where in this specification the term neutralize is used it means one or a combination of sterilizing and/or disinfection processes such as to render the contents of a biohazard neutralizing bag sufficiently inert or neutralized as to lower the biohazard nature of the waste to an acceptable level (e.g. “otherwise inactivated” so as not considered to be regulated medical waste or a hazardous material under Federal law). It does not necessarily mean that the contents of a biohazard neutralizing bag are sterilized. It will be appreciated that the concentration of any chemical sterilant, temperature of any vapor and the period of exposure will all affect the level of neutralization and specific combinations of parameters will be appropriate for different biohazards.

### BRIEF DESCRIPTION OF THE DRAWINGS

The invention will now be described by way of example with reference to the accompanying drawings in which:

FIG. 1 shows a perspective view of a neutralizing bag according to one embodiment;

FIG. 2 shows a plan view of the neutralizing bag shown in FIG. 1;

FIG. 3 shows an exploded view of the neutralizing bag shown in FIG. 1;

FIG. 4 shows a neutralizing bag including an inductively heated element in its base;

FIG. 5 shows a neutralizing bag including a resistive heating element in its base;

FIG. 6 shows a receptacle for holding a bag and facilitating connection of supply services;

FIG. 7 shows the receptacle holding the bag open when the foot pedal is depressed; and

FIG. 8 shows a detailed view of the bag coupling to the receptacle.

### DETAILED DESCRIPTION OF A PREFERRED EMBODIMENT OF THE INVENTION

In a preferred embodiment a multilayer non-porous bag is disclosed that can withstand sterilizing temperatures and be capable of retaining a vacuum for extended periods. This provides a method of rendering the infectious waste inert at point of containment—without the requirement for large autoclaves or specialist decontamination equipment. By including a built-in heating element the bag can perform an internal thermal and chemical sterilization process whilst fully sealing and containing the infectious waste within. Coupled with a pre-vacuum phase this provides a bag with the previously infectious contents rendered inert and vacuum packaged ready for safe transport by normal means. The process leaves the bag contents sealed in a vacuum so that any leak from the package is drawing air in rather than expelling the contents out. The combination thermal and chemical disinfection/sterilization method retains the sterilant chemical within the bag at the end of the process and this forms a further layer of safety by continuing to protect and decontaminate. A highly visible and identifiable colored dye may be added to the sterilant liquid to enable instant visual recognition of decontamination having occurred. Further processing of the bags may involve a macerating autoclave or incinerator following transportation to such a facility.

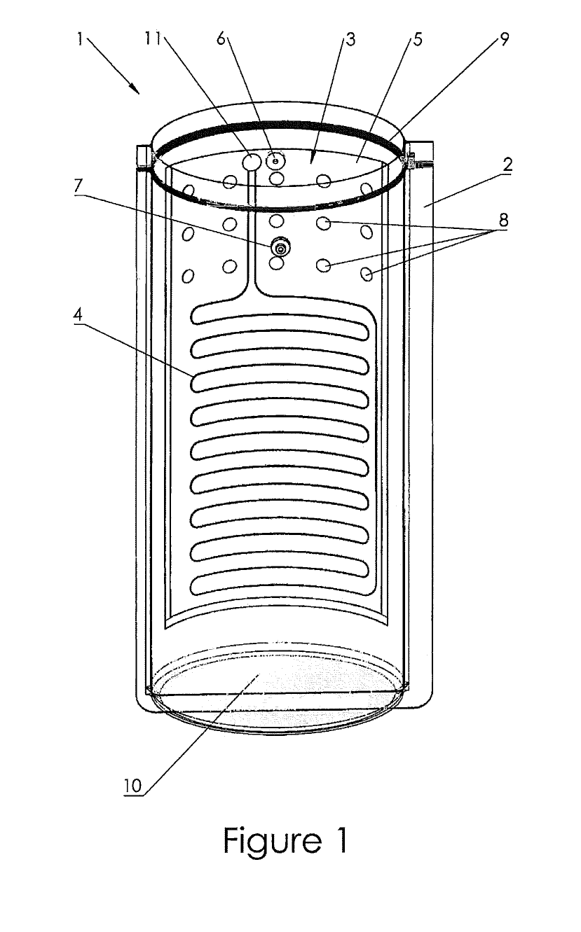

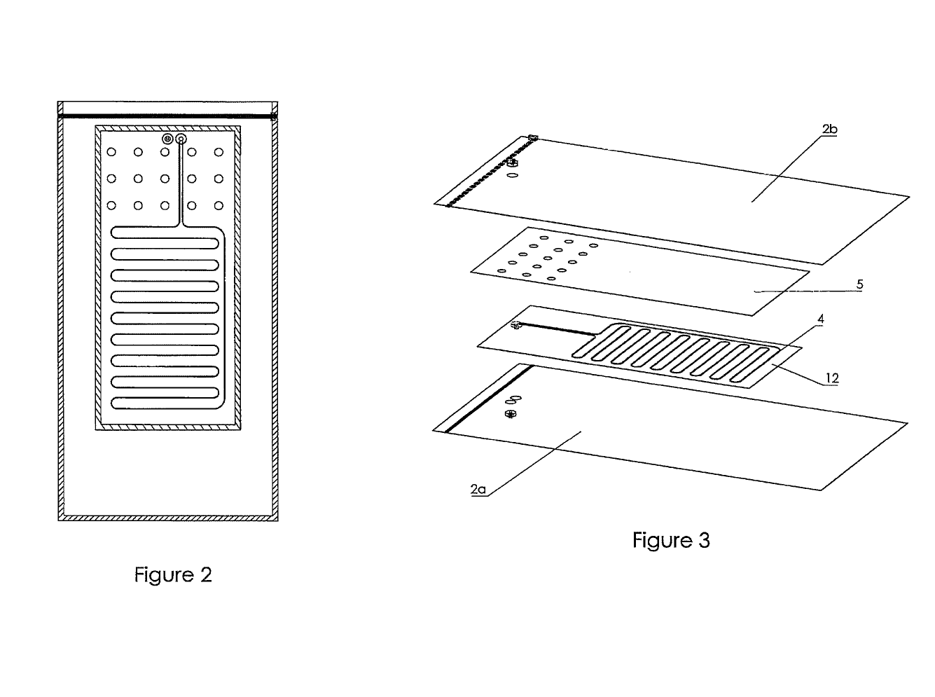

Referring to FIG. 1 there is shown a biohazard neutralizing bag 1 including a bag body 2 formed of a flexible material capable of withstanding a neutralization process performed within the bag having a sealable opening 3 sized to enable items to be neutralized to be placed within the bag body 2. A closure 9 is provided proximate the open end to allow the bag to be sealed after items to be neutralized have been placed in the bag. A closure in the form of a zip lock allows use by a triple gloved user and an adhesive seal tape may be applied to ensure a good seal. Alternatively the opening could be heat sealed or chemically bonded using an adhesive, cold welding etc. In one embodiment a chemical sealing agent may be provided in a narrow pouch extending along a zip and closure of the zip may release the chemical agent to seal the bag.

Suitable materials for the bag body may include polypropylene (PP), linear low-density polyethylene LDPE), high density polyethylene (HDPE) and Biax Nylon (BOPA) PET-AIOx/PP (Aluminium Oxide coated PET such as Barrialox) or EVOH. Multi-layer high seal strength may be employed. The exterior of the bag body may include an external antibacterial layer such as a copper or silver or titanium dioxide coating to reduce the risk of exterior surface contamination. The bag may be fibre reinforced for larger items or where greater integrity is required. The bag may conveniently be sized of the order of about 170 litres and able to withstand process temperatures greater than 134° C.

A further containment bag formed of canvas or a material capable of containing a rupture or bag failure may also be placed around the neutralizing bag when heating. This may assist in handling a heated bag and protect the integrity of the neutralizing bag.

An outlet 7 may be provided with an external screw or push fit coupling and a non-return valve to allow fluid to be evacuated from the bag body. Outlet 7 may include an integrated HEPA filter to prevent the release of biohazardous material. As well as allowing evacuation of the bag prior to neutralization outlet 7 also provides over pressure relief in case the neutralization process exceeds pressure limits and the HEPA filter prevents the release of biohazardous material.

In this embodiment a vapor generator in the form of a electrically resistive heating element 4 is provided on the inside of bag body 2. Terminals are provided to the exterior of bag body 2 at contact plates 11 to allow connection to an external electrical power source. The heating element 4 may be in the form of a printed thin or thick film polymer/polyimide/carbon or etched film aluminium/silver/copper transferred onto the bag surface or floating between pouch wall 5 and bag body 2.

Heating element 4 may be provided upon a material such as BoPET or polyimide to protect the bag body if the material of the bag body cannot withstand the direct heat of the heating element during neutralization. The heating element could be provided externally on the outside of bag body 2 if the bag body is formed of a suitable material that can conduct heat to the interior of bag body 2 and withstand process temperatures during neutralization.

In this embodiment a pouch is formed containing the element 4 between pouch wall 5 and bag body 2. The pouch wall 5 may be welded to bag body 2 so as to form a volume to receive a fluid to be vaporised proximate the element 4. An inlet 6 with a screw or push fit coupling allows fluid to be introduced into the pouch so that fluid to be vaporized is kept proximate the heat source to generate vapor. Apertures 8 may be provided in pouch wall 5 to allow the release of vapor into the bag body to neutralize its contents. As an alternative to this construction a porous material may be provided next to element 4 to retain a fluid to be vaporized proximate the element.

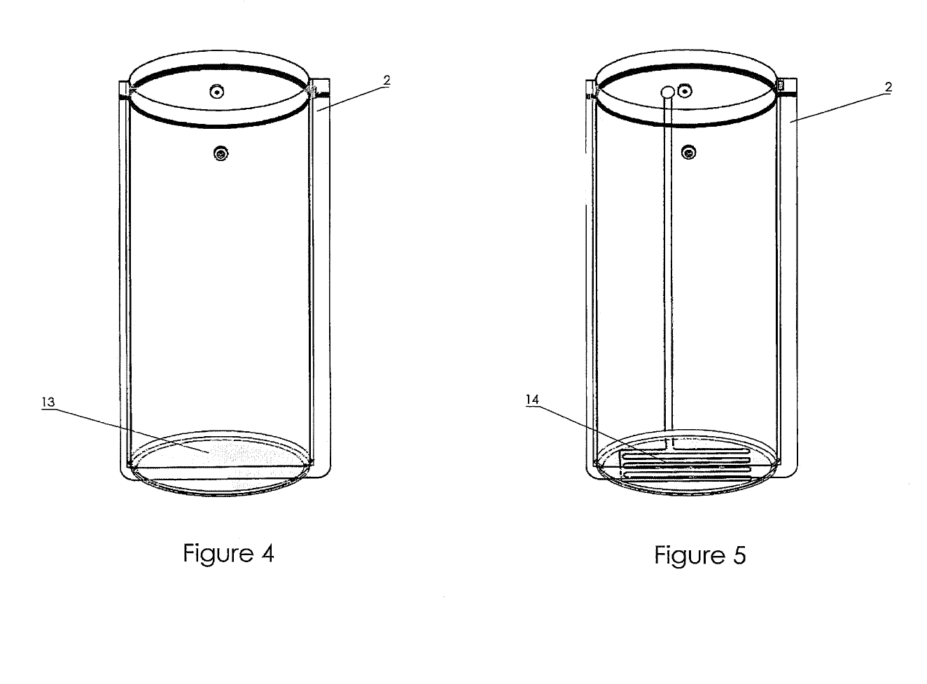

As shown in FIG. 4 a metal plate 13 may be placed in the base of bag body 2 and heated via an inductive coil positioned below it or as shown in FIG. 5 a resistive heating element 14 may be provided in the base of bag 2. These arrangements have the advantage that fluid to be vaporized will flow to the bottom of the bag proximate the heat source. The vapor generator may take other forms such as a small container containing a heating element and a fluid to be vaporised.

FIG. 3 shows an exploded view of the biohazard neutralizing bag shown in FIG. 1. bag body 2 is formed of sheets 2a and 2b welded along three edges. Heating element 4 is formed on a BoPET or polyimide support 12 and retained in a pouch formed by welding the edges of pouch wall 5 to bag wall 2a.

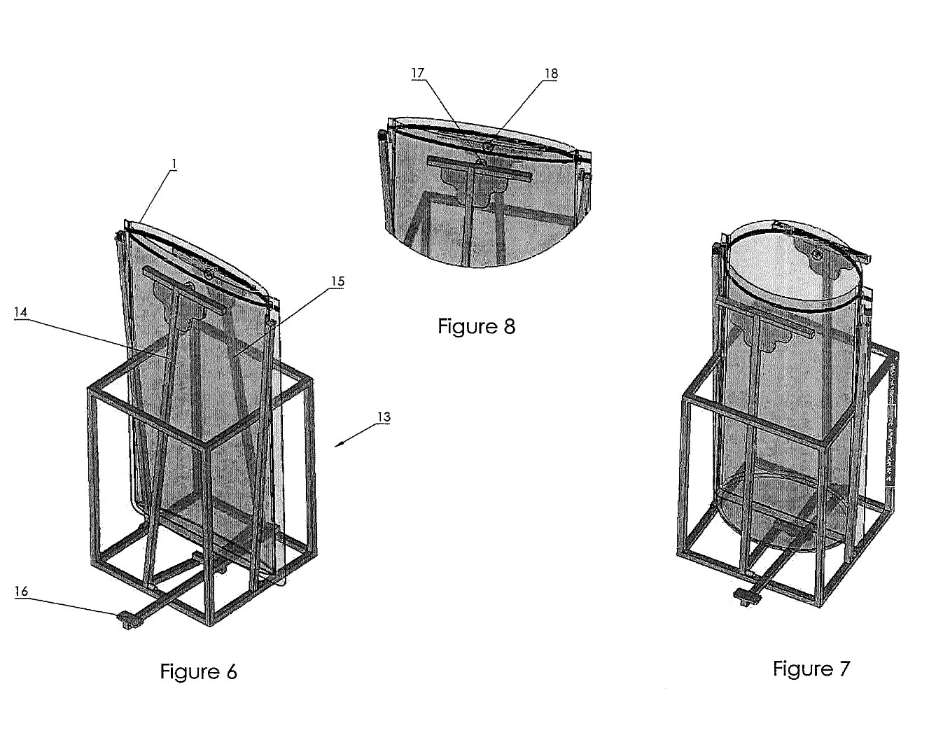

As shown in FIGS. 6 to 8, a frame 13 may be provided to support bag 1 connect services. A pair of arms 14 and 15 may be biased open by springs etc. with depression of pedal 16 causing the arms to separate as shown in FIG. 7. Arms 14 and 15 may include an evacuation line and sterilisation supply line respectively having complementary push fit couplings 17 and 18 to engage with inlet 6 and outlet 7. In use pedal 16 may be depressed to open arms 14 and 15 to the position shown in FIG. 7. A bag may be placed in position between the jaws and the pedal released to force complementary couplings 17 and 18 into engagement with inlet 6 and outlet 7 of bag 1. When an item is to be placed in bag 1 pedal 16 may be depressed to open the bag as shown in FIG. 7 and when the pedal is released the bag is closed. This arrangement facilitates closure as the bag mouth is held closed clips, hooks, clamps etc. may be used to secure the mouth of bag 1 to frame 13.

Once items have been placed within the bag closure 9 is closed to seal the bag.

An evacuation line (not shown) is then connected to outlet 7 via a screw or push fit coupling and fluid is evacuated from the bag. When the required vacuum level has been achieved for the correct duration the vacuum machine will indicate this and the vacuum apparatus is decoupled.

A fluid to be vaporized is then drawn into the pouch via inlet 6 due to the internal vacuum, although outlet 7 could potentially be adapted to introduce sterilant too. The fluid may simply be water to produce steam as a heat sterilant. Alternatively the fluid may consist of a vapor carrier and a chemical sterilant. The fluid may for example be a 5% formaldehyde WW solution for a Low Temperature Steam Formaldehyde process in which 8-16 mg of formaldehyde per litre of water is heated to 70-75 deg C. maintaining a relative humidity of 75-100%. Alternatively the fluid may be a water and hydrogen peroxide solution to perform a vaporized hydrogen peroxide sterilization process. Other chemical sterilants that may be employed include Ortho-phthaladehyde, Peracetic Acid, Glutaraldehyde, Potassium permanganate and Sodium Hypochlorite. A wide range of carrier fluids may also be employed. For a 170 litre bag approximately 2 litres of fluid may be required.

Whilst it is generally desirable to minimise size and weight of a bag in one embodiment the sterilant fluid (carrier fluid and/or chemical sterilant) may be sealed within the pouch and weakened regions of pouch wall 5 may rupture during processing to release sterilizing vapor.

It will be appreciated that if an heating element is provided in base 10 then a pouch is not required as carrier fluid and/or chemical sterilant will flow to the base 10 proximate the heating element to be vaporized during processing.

Once the bag is sealed and contains carrier fluid and/or chemical sterilant then the bag may be sprayed with a chemical sterilizer and placed within an insulated safety case. An exterior bag may be placed over the bag to provide protection from heat and penetration of bag. The heating element may then be connected to an electrical power supply and the carrier fluid and/or chemical sterilant proximate the heating element is vaporised. A plug, alligator type clips, spring or brush contact coupling may connect to contact plates 11. The heater element will vaporize the fluid forming a steam; low temperature steam/formaldehyde; or vaporized hydrogen peroxide mix which penetrates into the bag contents—thermally decontaminating and chemically sterilizing the load and rendering the bio-hazardous waste inert. This process will typically take around 30 minutes. The bag is then left to cool or placed in a cool-down facilitator which condenses the vaporized sterilant and returns the bag to a vacuum whilst retaining the chemical sterilant. The chemical sterilant continues to provide internal decontamination until the bag is incinerated or otherwise destroyed.

Where the fluid is only water then heating to about 100° C. will be effective to heat treat many pathogens such as Ebola, which is rendered inactive at 60° C. Where a chemical sterilant is employed the temperature needs to be sufficient to effectively carry the chemical sterilant by vapor. Where a chemical sterilant is carried by vapor neutralization is achieved by the chemical action of the chemical sterilant as well as the thermal sanitization of the carrier. Chemical sterilant sealed within bag continues to decontaminate and offers protection against recontamination during transport and storage.

The processing period may be controlled in a variety of ways. In the simplest from an operator may simply supply power for a prescribed time. A timer may of course be included in the power supply path to automatically control this. Alternatively processing may be continued until a visible indicator such as a thermochromic ink indicates that a required processing temperature has been achieved. More sophisticated control systems may be included in the supply to the bag that measure parameters such as processing temperature which controls power supply based on measured process parameters.

After processing the bag remains sealed and due to recondensation of vapor the interior of the bag will revert to near vacuum resulting in a reduced volume, reduced risk of rupture and providing an inert environment. The bag is then transferred to a disposal site.

In an alternative embodiment a bag without a heating element may be used and an external boiler may provide vaporized sterilant through an inlet to the bag. This simplifies the bag but requires an external boiler.

### Formaldehyde Steam.

Low-temperature steam with formaldehyde is used as a low-temperature sterilization method in many countries, particularly in Scandinavia, Germany, and the United Kingdom. The process involves the use of formalin, which is vaporized into a formaldehyde gas. A formaldehyde concentration of 8-16 mg/I may be generated at an operating temperature of 70-75° C. Low-temperature steam formaldehyde sterilization has been found effective against vegetative bacteria, mycobacteria, B. atrophaeus and G. stearothermophilus spores and Candida albicans. Reliable sterilization using formaldehyde is achieved when performed with a high concentration of gas, at a temperature between 60° and 80° C. and with a relative humidity of 75 to 100%.

### Vaporized Hydrogen Peroxide (VHP®).

VHP offers several appealing features that include rapid cycle time (e.g., 30-45 minutes); low temperature; environmentally safe by-products (H₂O, oxygen [O₂]); good material compatibility; and ease of operation, installation and monitoring. Vapor-phase hydrogen peroxide has been shown to possess significant sporicidal activity and has been found to be a highly effective method of eradicating MRSA, Serratia marcescens, Clostridium botulinum spores and Clostridium difficile from rooms, furniture, surfaces and/or equipment.

There is thus provided a biohazard bag and a method for neutralizing biohazards having the following advantages:
1. The bag is simple, inexpensive, compact and portable
2. The process has low infrastructure requirements making it suitable in a wide range of situations
3. The bag and process are safe and effective
4. a pre-vacuum stage removes air—which is particularly important when sterilizing porous loads or loads with entrapped air.
5. The method allows the use of either vapor only (e.g. steam) or vapor—carrier (e.g. steam formaldehyde) or vapor condensing (e.g. VHP).
6. The use of a vapor carrier and chemical sterilant provides thermal sterilization and a transport medium for the chemical sterilant which can result in an enhanced effect of the chemical sterilant at higher temperatures
7. Maintaining a vacuum within the bag:
   a. allows any lack of seal integrity to be easily identified
   b. reduces the volume to be transported and handled
   c. reduces the risk of rupture
   d. assists in keeping the contents of the bag inert.

The invention as described herein is open to modification as will be appreciated by those skilled in the art. For example, rather than perform as a sterilization package the packaging could be used as a retort or food cooking package but not limited to only these applications.

Other modifications and improvements to the invention will be apparent to the skilled person and will fall within the scope of the invention as it is intended.

1. A biohazard neutralizing bag including:
a. a bag body formed of a flexible impermeable material capable of withstanding a neutralization process performed within the bag having a sealable opening sized to enable items to be neutralized to be positioned within the bag body; and
b. a vapor generator which, when activated, heats a fluid within the bag body to generate vapor to neutralize the contents of the bag.

2. A biohazard neutralizing bag as claimed in claim 1 including an electrical heating element within or secured to the bag body having terminals for connection to an external electric power supply.

3. A biohazard neutralizing bag as claimed in claim 2 wherein the bag has a base and side walls extending from the base and the electrical heating element is provided proximate the base of the bag.

4. A biohazard neutralizing bag as claimed in claim 2 wherein the bag includes an internal pouch for receiving liquid sterilant.

5. A biohazard neutralizing bag as claimed in claim 4 wherein the electrical heating element is contained within the pouch.

6. A biohazard neutralizing bag as claimed in claim 4 wherein a sterilant inlet is provided in a wall of the bag body proximate the pouch.

7. A biohazard neutralizing bag as claimed in claim 4 wherein the pouch include outlets in the pouch for releasing vapor into the interior of the bag body.

8. A biohazard neutralizing bag as claimed in claim 1 including a fluid outlet in a wall of the bag body for removing fluid from the bag body.

9. A biohazard neutralizing bag as claimed in claim 8 including a HEPA filter in its flow path.

10. A biohazard neutralizing bag as claimed in claim 8 including a one way valve allowing only fluid removal from the bag body.

11. A biohazard neutralizing bag as claimed in claim 1 wherein a zipper is provided proximate the sealable opening to seal the sealable opening when closed.

12. A biohazard neutralizing bag as claimed in claim 1 wherein a pressure adhesive seal is provided proximate the sealable opening to seal the sealable opening when closed.

13. A biohazard neutralizing bag as claimed in claim 1 wherein the region proximate the sealable opening is formed of heat sealable material to facilitate sealing of the sealable opening.

14. A biohazard neutralizing bag as claimed in claim 1 containing a chemical sterilant.

15. A biohazard neutralizing bag as claimed in claim 14 wherein the sterilant is selected from one of Hydrogen Peroxide, Formaldehyde Ortho-phthaladehyde, Peracetic Acid and Sodium Hypochlorite.

16. A biohazard neutralizing bag as claimed in claim 1 containing a vapor producing liquid selected from water, alcohol and methylated spirits.

17. A method of neutralizing one or more items comprising:
a. placing one or more items to be neutralized within a sealable bag having a sealable opening for receiving items to be neutralized, an outlet for the removal of fluid and an integrated sterilant vapor generator;
b. sealing the sealable opening;
c. evacuating the bag via the outlet; and
d. actuating the vapor generator to generate vapor such as to produce conditions sufficient to neutralize the one or more items.

18. A method as claimed in claim 17 wherein the bag includes an inlet for introducing into the bag a fluid to be vaporised and fluid is introduced into the bag prior to actuating the vapor generator.

19. A method as claimed in claim 17 wherein the vapor generator includes an electrical heater element which is actuated by connecting electrical leads exiting the bag to an electrical power source.

20. A method as claimed in claim 17 wherein the bag includes a pouch containing the vapor generator and the fluid is introduce into the pouch prior to activating the vapor generator.

21. A method as claimed in claim 17 wherein the vapor generator boils water to produce steam.

22. A method as claimed in claim 17 wherein a carrier fluid is vaporised by the vapor generator to carry a chemical sterilant throughout the bag to effect neutralization.

23. A method as claimed in claim 22 wherein the sterilant is selected from one of Hydrogen Peroxide, Formaldehyde Ortho-phthaladehyde, Peracetic Acid and Sodium Hypochlorite.

24. A biohazard neutralizing system
a. a bag body formed of a flexible material capable of withstanding a steam sterilization process performed within the bag having:
i. a sealable opening sized to enable items to be neutralized to be positioned within the bag body; and
ii. at least one port for evacuation of the bag and the supply of vaporized sterilant; and
b. a portable vapor generator adapted to supply vaporized sterilant to the bag via the sealable port.
